(19) United States
(12) Patent Application Publication
(10) Pub. No.: US 2012/0197299 A1
(43) Pub. Date: Aug. 2, 2012

(54) SPINE SURGERY METHOD AND IMPLANT DEPLOYMENT

(76) Inventor: Henry F. Fabian, JR., Steamboat Springs, CO (US)

(21) Appl. No.: 13/016,410

(22) Filed: Jan. 28, 2011

(51) Int. Cl. A61B 17/88; A61B 17/56

(52) U.S. Cl. 606/279; 606/86 A

(57) ABSTRACT

A surgical inserter may include an implant deployment mechanism that is adjustable by the surgeon to deploy an implant within the vertebral space and may include a deployment indicator that provides a visual indication to the surgeon regarding the status of implant deployment within the vertebral space.

### I. BACKGROUND OF THE INVENTION

A. Field of Invention

This invention pertains to the art of methods and apparatuses regarding spine surgery and more specifically relates to surgical procedures and instruments used to deploy an implant within a vertebral space.

B. Description of the Related Art

The volume of spinal surgeries to treat degenerative disc and facet disease has steadily increased over the past decade, fueled by population demographics and advancements in diagnostic and instrumentation adjuncts. Improvements in intraoperative radiological imaging and surgical technique have generated a great deal of interest in applying minimally invasive surgical (MIS) techniques to spinal applications. As in other surgical subspecialties, it is hoped such minimally invasive techniques applied to spinal surgery will result in less soft tissue trauma, less operative blood loss, reduced operative time, faster recovery periods and lower costs.

Known spinal surgical techniques, though generally working well for their intended purposes, have been adopted from traditional open surgical (non-MIS) techniques. As a result, known spinal surgical methods, instrumentation and interbody implants have limitations. One limitation is that the physical components are relatively large and bulky. This reduces surgeon visualization of the surgical site. Another limitation of known spinal surgical methods is that known surgical tools and implants are cumbersome and difficult to maneuver within the limited surgical space available. The limitations of current instrumentation in MIS spine surgery are noted particularly with regards to interbody fusion surgery.

The present invention provides methods and apparatuses for overcoming these limitations by providing surgical instrumentation that allows for minimally invasive spinal surgery and that provides for precise movement, placement and deployment of an implant into the vertebral space.

### II. SUMMARY OF THE INVENTION

According to one embodiment of this invention, a device may comprise: (A) an inserter for use in inserting an associated implant into a vertebral space, the inserter comprising: a handle for use by a surgeon; (B) an implant gripping mechanism supported to the inserter and adjustable by the surgeon to grip and to release the associated implant within the vertebral space; (C) an implant deployment mechanism supported to the inserter and adjustable by the surgeon to deploy the associated implant within the vertebral space; and, (D) a deployment indicator supported to the inserter that provides a visual indication to the surgeon regarding the status of implant deployment within the vertebral space.

According to another embodiment of this invention, a method may comprise the steps of: (A) providing an implant made to be placed into a vertebral space and comprising a first member and a second member movably attached to the first member; (B) providing a surgical inserter; (C) providing a cable operatively attached to the surgical inserter and operatively attached to at least one of the first or second members; (D) preparing the vertebral space to receive the implant; (E) inserting the implant into the vertebral space in a non-deployed condition; and, (F) applying tension to the cable to: (1) move the second member with respect to the first member to deploy the implant within the vertebral space; and, (2) intentionally cause the cable to fail.

According to yet another embodiment of this invention, a surgical system may comprise: (A) an implant made to be placed into a vertebral space and comprising: a first member and a second member movably attached to the first member; (B) an inserter for use in inserting the implant into the vertebral space, the inserter comprising: a handle mechanism for use by a surgeon; (C) an implant gripping mechanism supported to the handle mechanism and adjustable by the surgeon to grip and to release the implant within the vertebral space; and, (D) an implant deployment mechanism supported to the handle mechanism and comprising: a drive shaft that: is attachable to the implant; and, can be rotated by the surgeon to deploy the associated implant within the vertebral space by moving the first member of the implant with respect to the second member of the implant.

Numerous benefits and advantages of the invention will become apparent to those skilled in the art to which it pertains upon a reading and understanding of the following detailed specification.

### III. BRIEF DESCRIPTION OF THE DRAWINGS

The invention may take physical form in certain parts and arrangement of parts, embodiments of which will be described in detail in this specification and illustrated in the accompanying drawings which form a part hereof and wherein:

FIG. 1 is a side perspective view of a spinal segment showing a vertebral space defined by the intradiscal space usually occupied by a disc between two adjacent vertebral bodies.

FIG. 2 is a side perspective view of a spinal segment showing a vertebral space defined by the space usually occupied by a vertebral body and its two adjacent discs.

FIG. 3 is a side view of an inserter according to one embodiment of this invention.

FIG. 4 is a back perspective view of the inserter shown in FIG. 3.

FIG. 5 is a side perspective view of a first handle member.

FIG. 6 is a side perspective view of a second handle member.

FIG. 7 is a side view showing a grip shaft received in the second handle member.

FIG. 8 is a side view showing the grip shaft received in the first handle member.

FIG. 9 is side view of gripper assembly.

FIG. 10 is a side view of gripper assembly shown in FIG. 9 but with the gripper assembly rotated about 90 degrees about its longitudinal axis.

FIG. 11 is a perspective end view of a grip shaft.

FIG. 12 is a perspective end view of a gripper.

FIG. 13 is a perspective opposite end view of the gripper shown in FIG. 12.

FIG. 14 is a perspective view of a contact member.

FIG. 15 is a perspective end view of a gripper and a contact member.

FIG. 16 is a side view of a grip activator shown received within reception tabs formed on the handle member.

FIG. 17 is a side perspective view of a grip activator.

FIG. 18 is a side perspective view showing a grip activator as transparent so that a coupler is visible.

FIG. 19 is a side perspective view of the coupler shown in FIG. 18.

FIG. 20 is a side perspective view similar to that shown in FIG. 19 but with the coupler rotated about 180 degrees about its longitudinal axis.

FIG. 21 is a top perspective view of an implant.

FIG. 22 is a side perspective view of an implant held by a gripper.

FIG. 23 is a top perspective view of an implant in a deployed condition.

FIG. 24 is a side perspective view of the posts from the implant of FIG. 23.

FIG. 25 is an end perspective view of the implant posts shown in FIG. 21.

FIG. 26 is a side view of an inserter with some components removed.

FIG. 27 is a side view of an inserter with some components removed.

FIG. 28 is a close-up side view of an inserter with some components removed.

FIG. 29 is a side view of a first drive rod interconnected with a compression spring 510. The use of the compression spring 510

FIG. 30 is a side view similar to that shown in FIG. 29 but also showing a second drive rod interconnected with the compression spring.

FIG. 31 is a side perspective view of a coupling.

FIG. 32 is an end perspective view of the coupling shown in FIG. 31 but shown as transparent.

FIG. 33 is a side view similar to that shown in FIG. 30 but also showing a coupling and a bushing as transparent.

FIG. 34 is a side perspective view of a second drive rod.

FIG. 35 is an end perspective view of a screw.

FIG. 36 is a view similar to that shown in FIG. 35 but showing the screw from the opposite end.

FIG. 37 is a side perspective view of a screw received in implant posts.

FIG. 38 is a side perspective view of a clip.

FIG. 39 is a view similar to that shown in FIG. 38 but showing the clip from the opposite side.

FIG. 40 is a top view of a portion of an inserter.

FIG. 41 is a close-up side perspective view of a portion of the inserter shown in FIG. 40.

FIG. 42 is a close-up side perspective view of a portion of the inserter.

FIG. 43 is a side perspective view of a deployment indicator.

FIG. 44 is a side view of an inserter according to another embodiment of this invention.

FIG. 45 is a top perspective view of an implant.

FIG. 46 is a side perspective view of some of the implant posts shown in FIG. 45.

FIG. 47 is a schematic representation of a cable according to one embodiment of this invention.

FIG. 48 is a schematic representation of a cable according to another embodiment of this invention.

FIG. 49 is a top view schematic representation of a deployment mechanism according to another embodiment of the invention.

FIG. 50 is a view similar to that shown in FIG. 49 but showing the implant in a deployed condition.

FIG. 51 is an end view schematic representation of an implant according to one embodiment of the invention.

FIG. 52 is an end view schematic representation of an implant according to another embodiment of the invention.

FIG. 53 is a top view schematic representation of the implant shown in FIG. 52 in a deployed condition.

### IV. DETAILED DESCRIPTION OF INVENTION

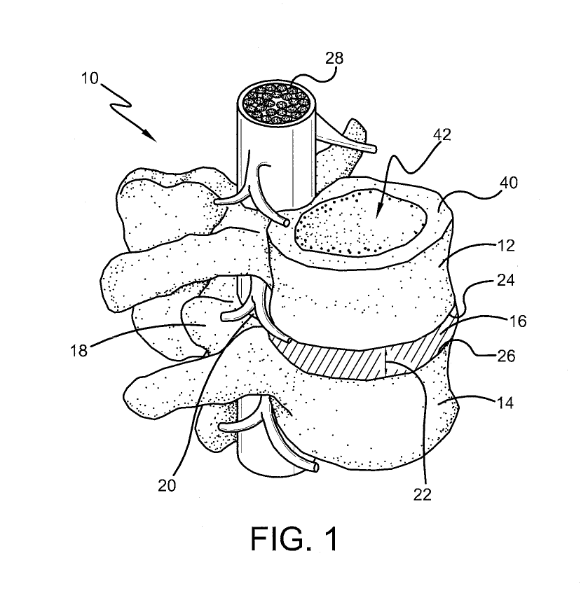

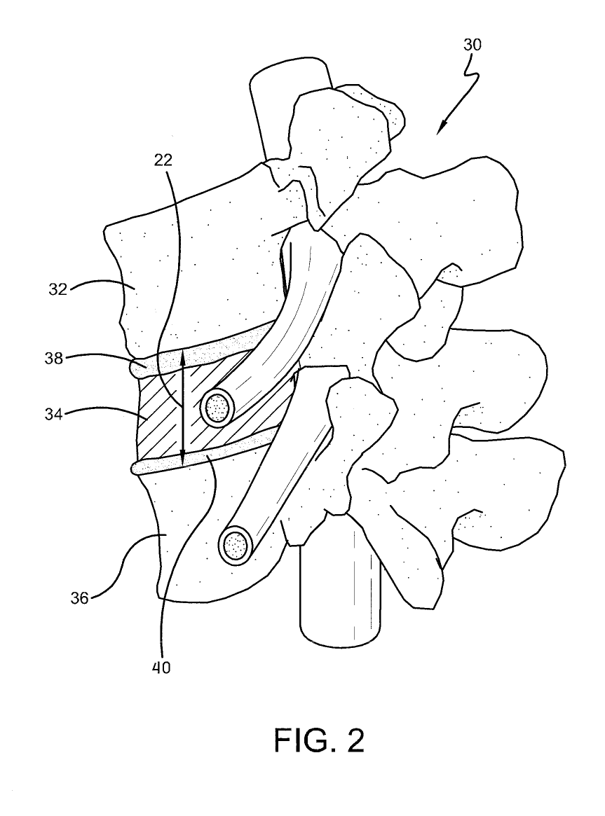

Referring now to the drawings wherein the showings are for purposes of illustrating embodiments of the invention only and not for purposes of limiting the same, a surgical inserter according to various embodiments of this invention may be used to insert and deploy an implant 100 into a vertebral space 22. By vertebral space it is meant the space in a spinal column where the implant 100 will be placed. In one embodiment, shown in FIG. 1, a spinal segment 10 is made up of two vertebrae 12, 14 attached together by ligaments with a disc 16 separating them. Facet joints 18 fit between the two vertebrae 12, 14 and allow for movement. The neural foramen 20 between the vertebrae 12, 14 allow space for the nerve roots to travel freely from the spinal cord 28 to the body. If it is required to remove the disc 16 and replaced it with an implant 100, the space occupied by the disc 16, the intradiscal space between the two adjacent vertebral bodies 12, 14, defines the vertebral space 22. In another embodiment, shown in FIG. 2, a spinal segment 30 is made up of three vertebrae 32, 34, 36 attached together by ligaments. If it is required to remove the middle vertebra 34 (it is shown diseased) along with the adjacent discs 38, 40, such as may be required because of a corpectomy defect, and replaced them with an implant 100, the space between the two outer vertebral bodies 32, 36, defines the vertebral space 22. It should be understood that these are simply two non-limiting examples of the vertebral space 22 into which an implant 100 can be inserted according to this invention because any vertebral space chosen with the sound judgment of a person of skill in the art can be used. As the components and operation of a spinal column are well known to those of skill in the art, further detail will not be provided here.

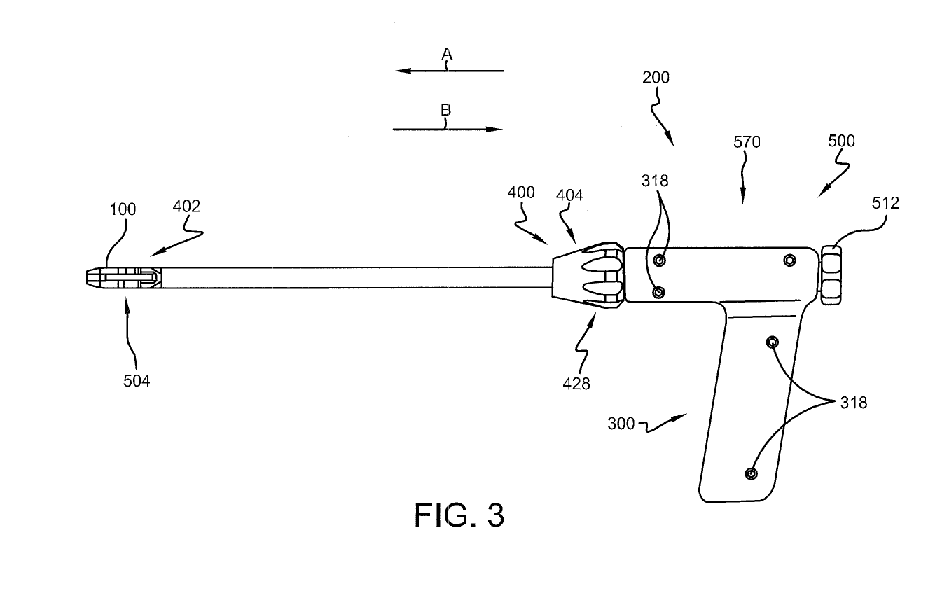

With reference now to FIG. 3, an inserter 200 according to one embodiment of this invention may include a handle mechanism 300, an implant gripping mechanism 400, and an implant deployment mechanism 500. Each of these mechanisms will be described in more detail below. Note that throughout this patent the term “proximal” shall refer to direction A as shown in FIG. 3 (toward the handle end of the inserter) and the term “distal” shall refer to direction B as shown in FIG. 3 (toward the implant end of the inserter). These terms are not used to limit this invention in any way but only to provide a direction reference.

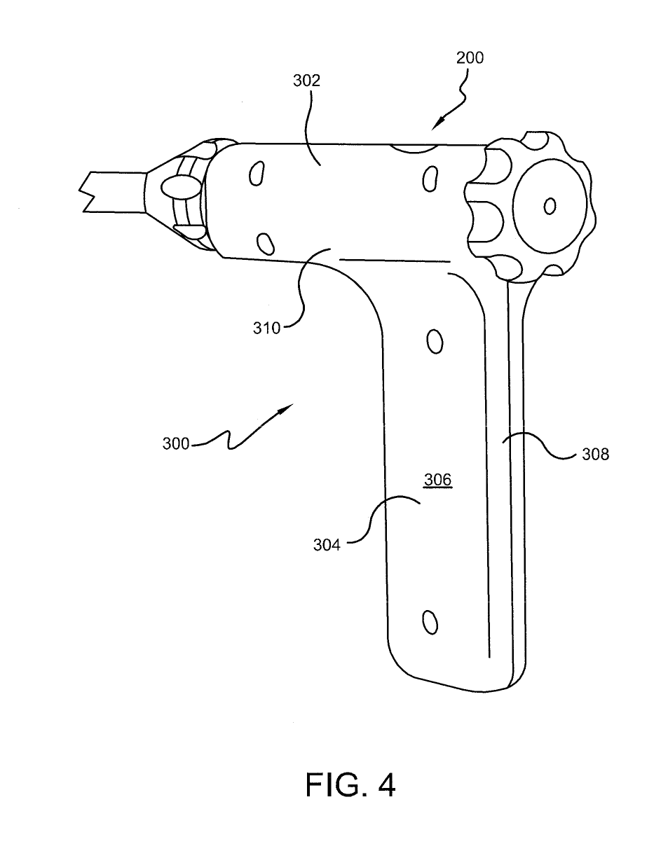

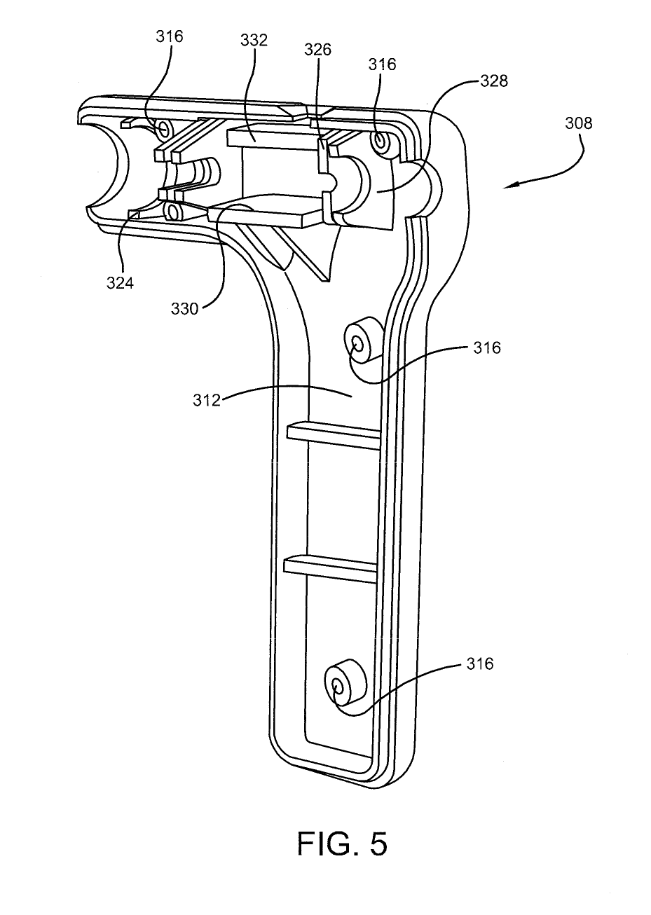

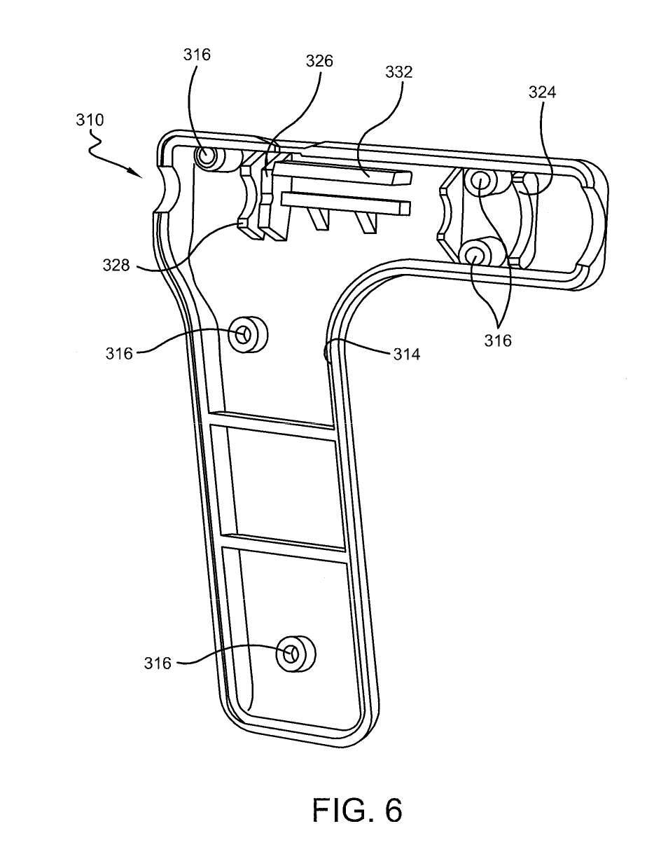

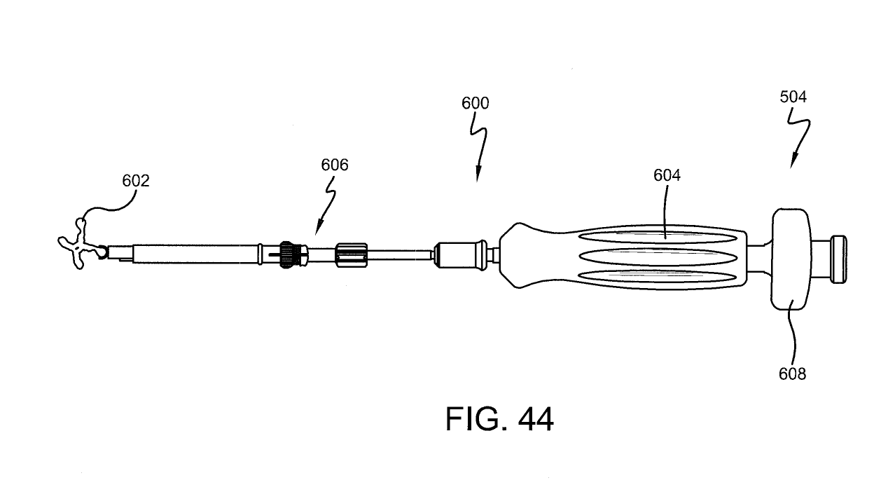

With reference now to FIGS. 3-6 the handle mechanism 300 may include a main body 302 and a handle 304 that extends from the main body 302 and that may be held by the surgeon and used to manipulate the inserter 200 during surgery. The handle can be of any size and shape chosen with the sound judgment of a person of skill in the art. In one embodiment, shown in FIG. 44, a handle mechanism 604 may be generally cylindrical in cross-section. For the embodiment shown in FIGS. 3-6, the handle 304 may be shaped and sized similar to a pistol handle. Such a pistol handle provides additional gripping surface for the surgeon and also provides for a different hand orientation for the surgeon. This hand orientation, similar to that provided on a handgun pistol, may assist the surgeon by improving the ergonomics of the inserter 200 and/or by improving the visibility of the surgical area and surgical instruments. The handle 304 may have a textured region 306 to improve the grip for the surgeon. While the handle mechanism 300 may be formed and attached together in any manner chosen with the sound judgment of a person of skill in the art, for the embodiment shown the handle mechanism 300 is formed with first and second handle members 308, 310 that attach to each other. In one embodiment, the first handle member 308 has a lip 312 that is received in a corresponding recess 314 formed in the second handle member 310. If desired, the handle members 308, 310 may have one or more openings 316 that are aligned and that receive connectors 318 to better secure the handle members 308, 310 together and to hold the other components to the handle mechanism 300, as will be discussed further below.

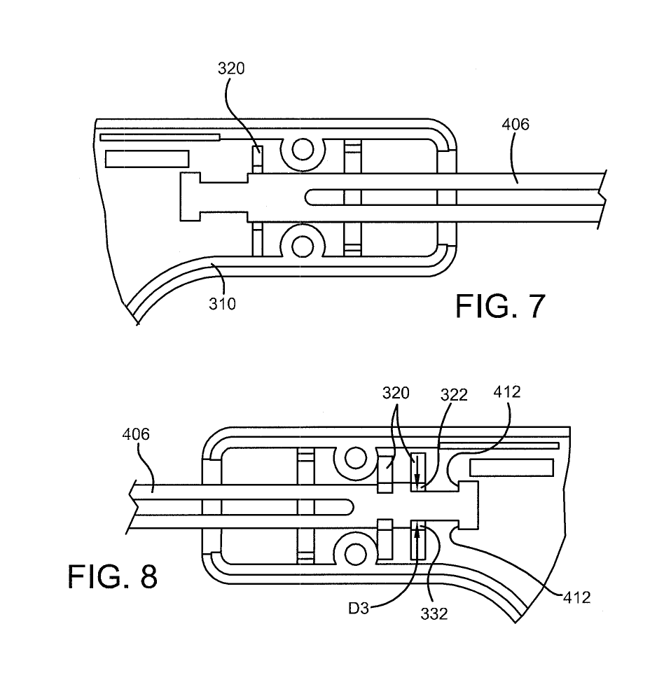

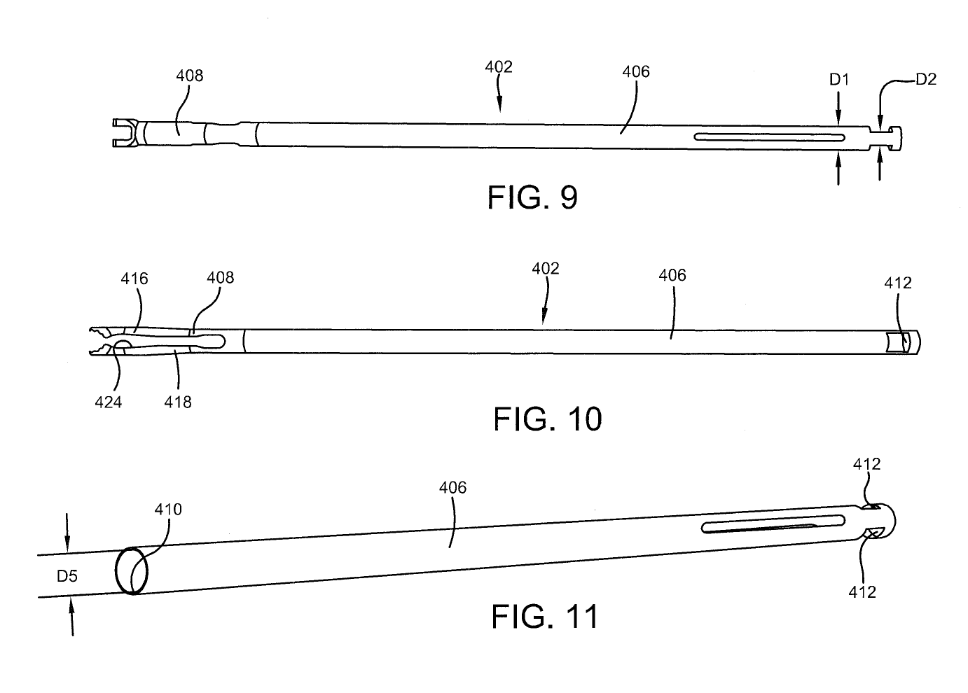

With reference now to FIGS. 3, and 5-11, the implant gripping mechanism 400, which is used to grip and release the implant 100, will now be described. The implant gripping mechanism 400 may include a gripper assembly 402 and a grip activator 404. The gripper assembly 402 may include a grip shaft 406 and a gripper 408. The grip shaft 406 may be generally cylindrical and may have an opening 410 throughout its length. The proximal end of the grip shaft 406 may attach to the handle mechanism 300. The grip shaft 406 may have an outside diameter D1 and may have a pair of cut-out sections 412 on opposite sides of the proximal end of the grip shaft 406, as shown, to create an effective outside diameter D2 that is less than outside diameter D1. In one embodiment, the proximal end of the grip shaft 406 is received within shaft reception tabs 320 formed on the handle members 308, 310, as shown. At least one of the shaft reception tabs 320 may have extension members 322 separated by a distance D3 that is less than the outside diameter D1 but greater than the diameter D2. In this way, the proximal end of the grip shaft 406 remains attached to the handle mechanism 300 via the shaft reception tabs 320.

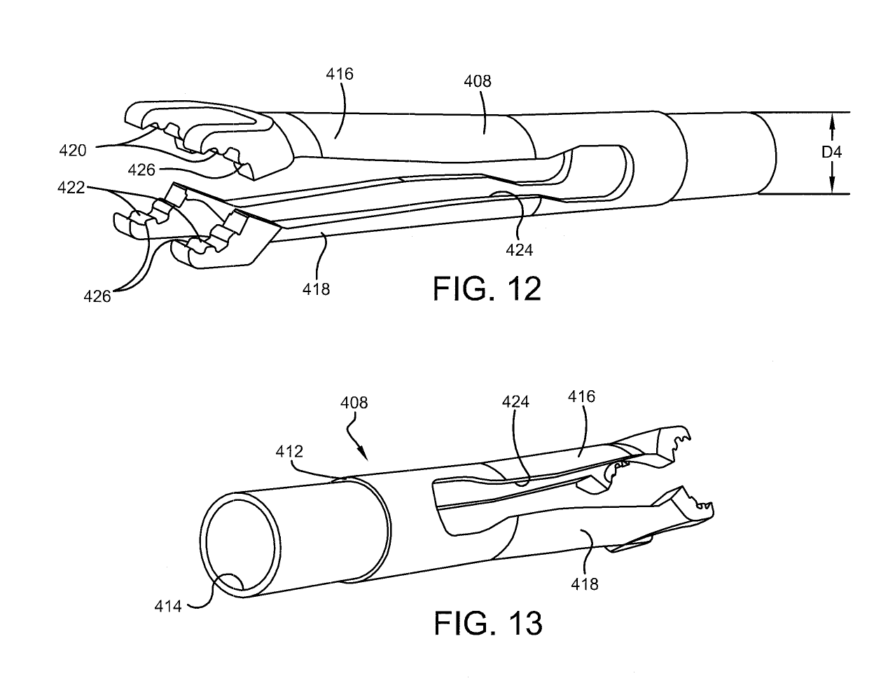

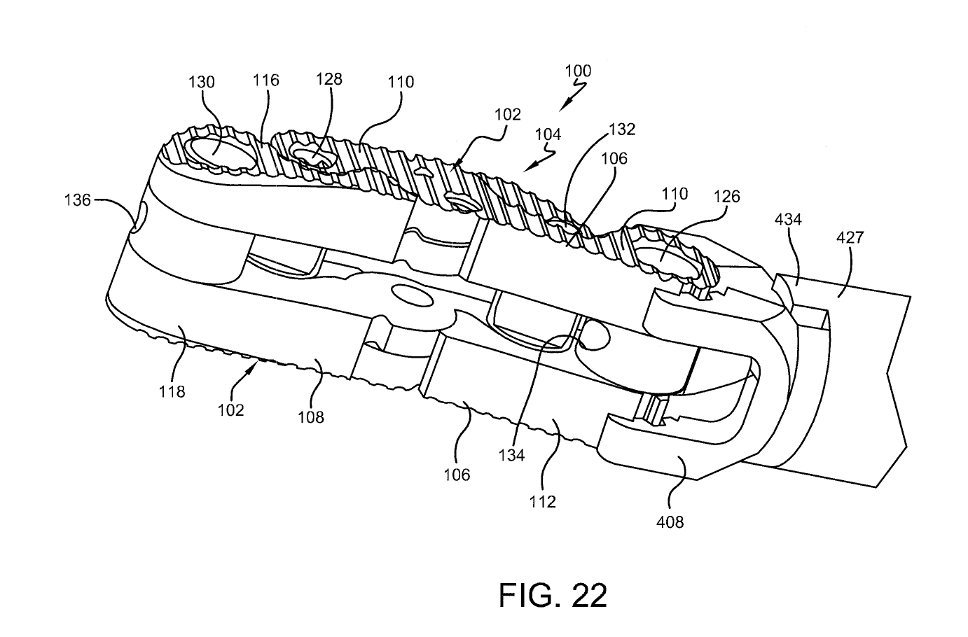

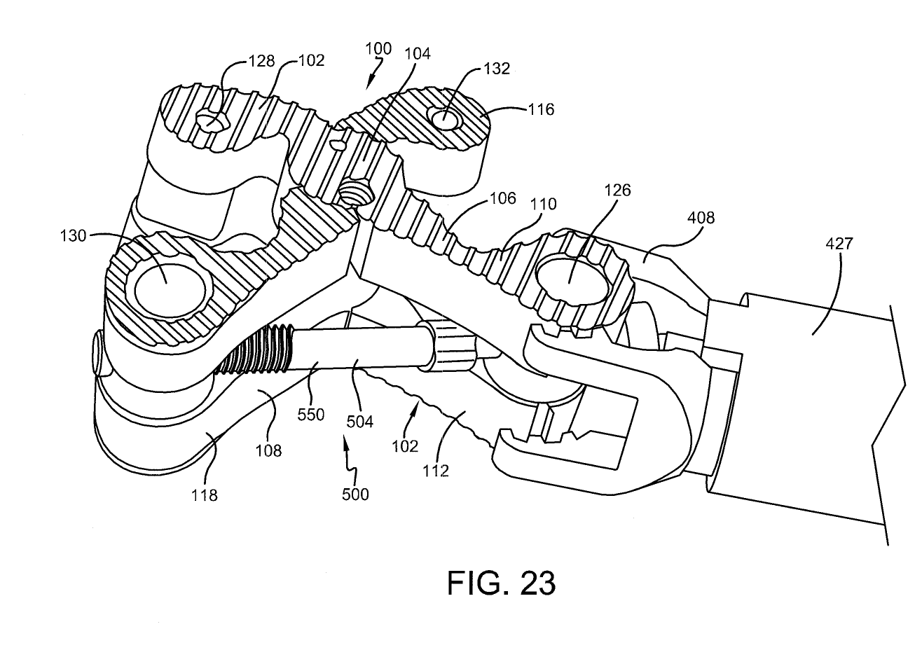

With reference now to FIGS. 3 and 9-13, the gripper 408 may have a generally cylindrical proximal end with an opening 414 and an outside diameter D4 that is smaller than the inside diameter D5 of the distal end of the grip shaft 406. In this way the proximal end of the gripper 408 is received within the distal end of the grip shaft 406. The gripper 408 may have an edge 412 to limit the distance that the grip shaft 406 can be extended over the gripper 408. The distal end of the gripper 408 may be used to both grip and release the implant 100. A pair of arms 416, 418, forming a V-shape, may extend outwardly and end with a pair of hands 420, 422, respectively, as shown. There may be a space 424 between the arms 416, 418 which can be narrowed as will be described further below. Each hand 420, 422 may have a contact surface 426 that is used to physically contact a surface of the implant 100. The contact surface 426 of each hand portion may be textured, as shown, to improve the gripping characteristics of the hands 420, 422. The gripper 408 may be used to grip any portion of the implant chosen with the sound judgment of a person of skill in the art. One embodiment is shown in FIGS. 22 and 23.

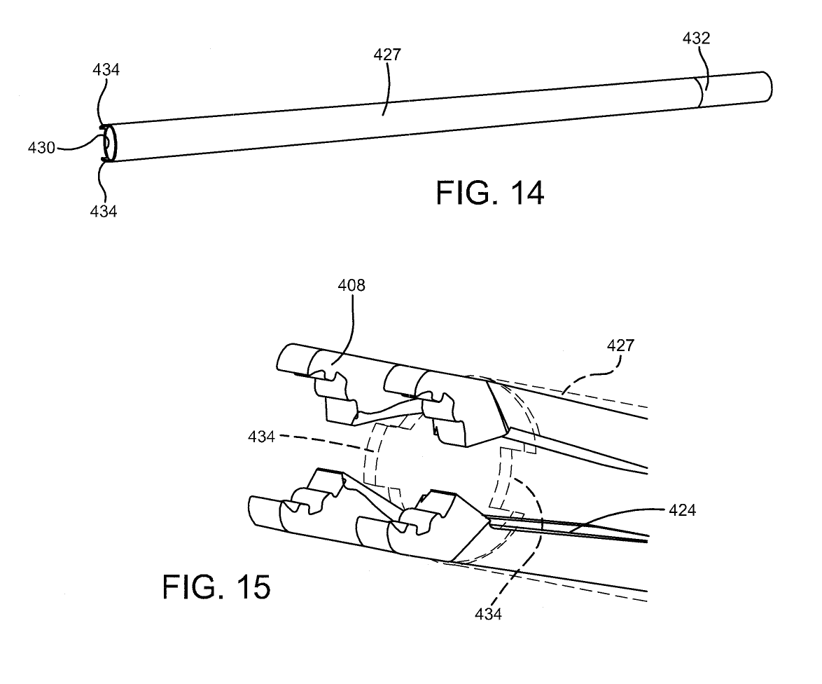

With reference now to FIGS. 3 and 12-15, the grip activator 404 may be used by the surgeon to activate the gripper 408 to grip and/or release the implant 100. The grip activator 404 may include a contact member 427 that contacts the gripper 408 and an activation member 428 that is operated by the surgeon to cause the contact member 427 to move into contact with the gripper 408. The contact member 427 may be generally cylindrical and may have an opening 430 throughout its length. This opening 430 may be positioned substantially in the radial center of the contact member 427, as shown. The proximal end of the contact member 427 may have a reduced outside diameter portion 432 used as described below. The distal end of the contact member 427 may have at least one extension 434 (two shown) that is positioned along one side of the space 424 to permit maximum distal relative motion of the contact member 427 with respect to the gripper 408 and thus to permit full gripping force for the gripper 408.

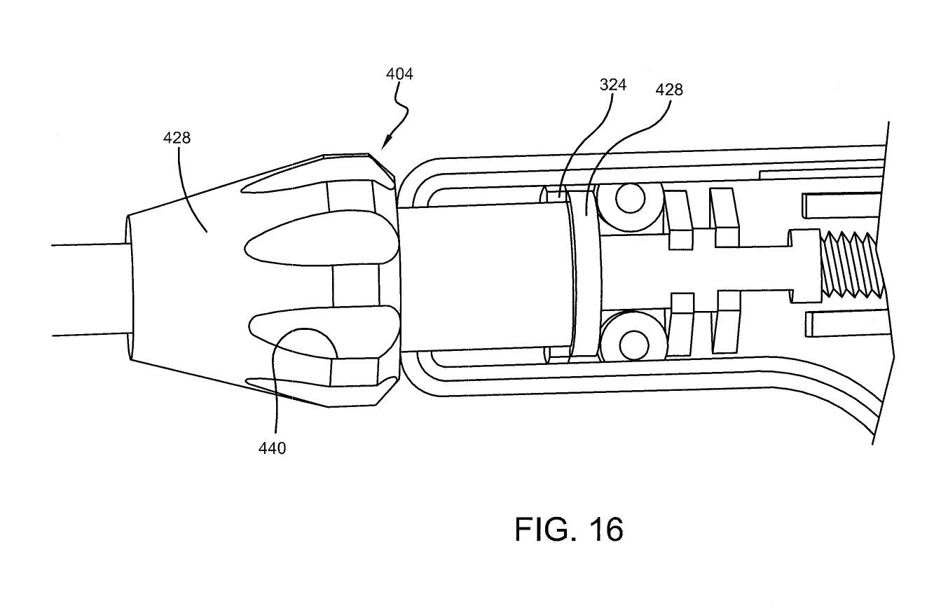

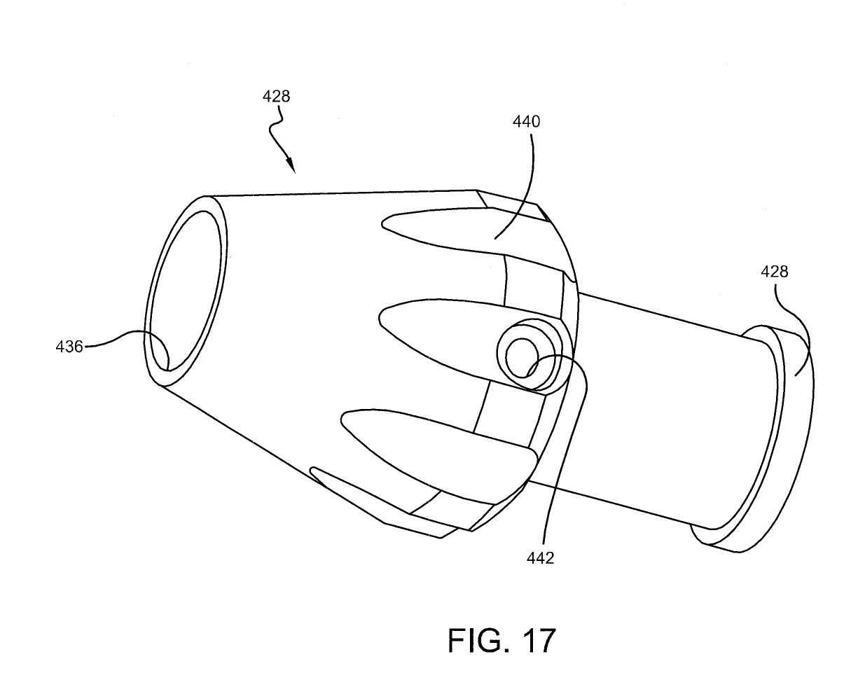

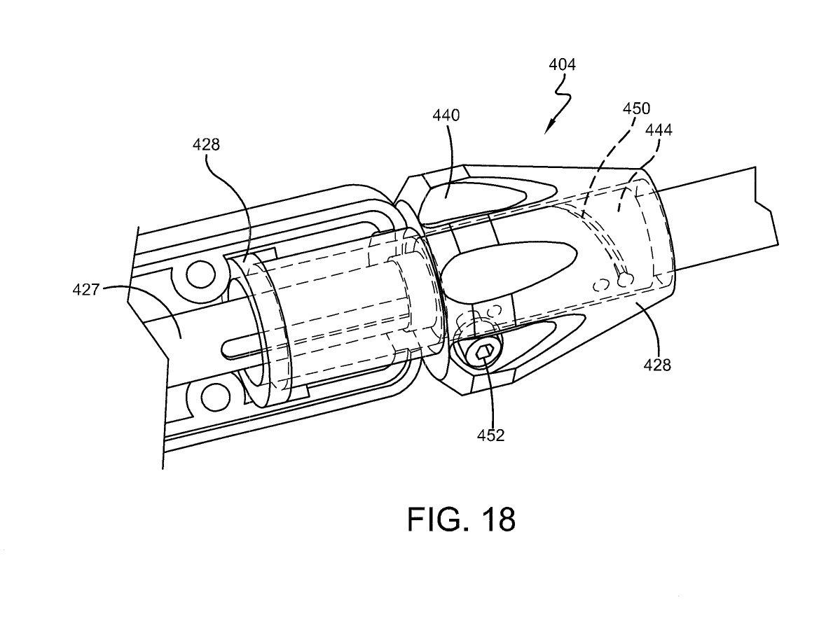

With reference now to FIGS. 3, 5-6 and 16-18, the activation member 428 may be generally cylindrical and may have an opening 436 throughout its length. The proximal end of the activation member 428 may be attached to the handle mechanism 300. For the embodiment shown, the proximal end has a ridge 428 for this purpose. The ridge 428 has an outside diameter greater than the outside diameter of the juxtaposed portion of the activation member 428, as shown. This ridge 428 may be positioned on the proximal side of a pair of shaft reception tabs 324 formed on the handle members 308, 310, as shown. With this arrangement, the activation member 428 can be rotated with respect to the handle mechanism 300 but cannot move longitudinally with respect to the handle mechanism 300. The distal end of the activation member 428 may be positioned outside of the handle mechanism 300 and may have an outer surface 440 that has a textured region to improve the grip for the surgeon. The activation member 428 may have a hole 442 for a purpose to be discussed below.

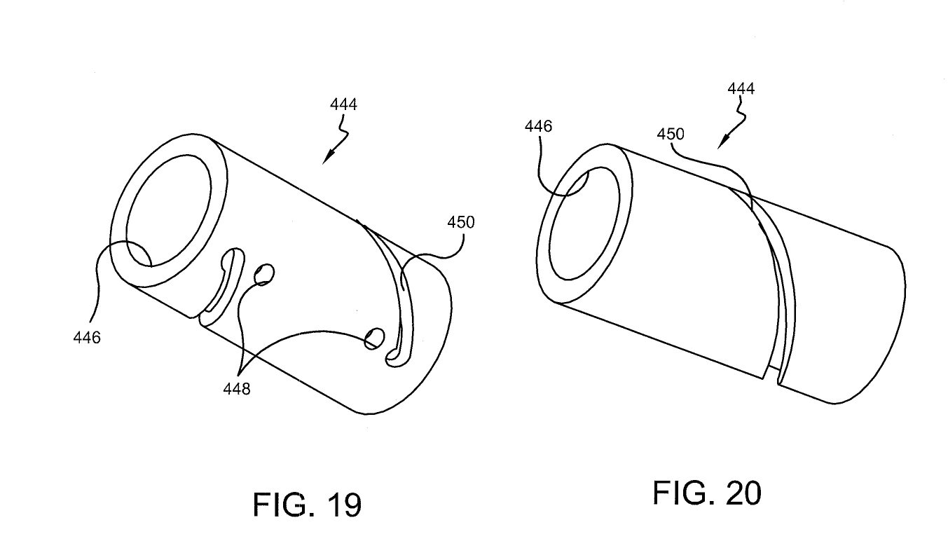

With reference now to FIGS. 3, and 16-20, a coupler 444 may be used to attach the activation member 428 to the contact member 427. The coupler 444 may be generally cylindrical and may have an opening 446 throughout its length. The coupler 444 may also have at least one hole 448 (two shown) through which a connector (not shown) may be inserted to fixedly attach the coupler 444 to the contact member 427. The contact member 427 may be positioned within the opening 446 just below the coupler 444 and thus is easily accessible through the hole 448. A groove 450 may be formed on the outer surface of the coupler 444 and may extend from the proximal end to the distal end. The groove 450 may also make one revolution (360 degrees) around the coupler 444 from one end to the other, as shown. It is also contemplated to form the groove 450 to make less than revolution or more than one revolution, as determined by a person of skill in the art. A pin 452, such as a machine screw, may inserted through the hole 442 in the activation member 428 and may extend into the groove 450. With this arrangement, when the surgeon rotates the activation member 428 with respect to the handle mechanism 300, the pin 452 is forced to move along the groove 450 to thereby cause the coupler 444 and thus the contact member 428 to move relative to gripper 408. Thus, by rotating the activation member 428, the surgeon can easily grip the implant 100 (by rotating the activation member 428 in one direction) and release the implant 100 (by rotating the activation member 428 in the opposite direction).

With reference now to FIGS. 3 and 21-23, embodiments of the implant deployment mechanism 500, which is used to deploy the implant 100, will soon be described. First, however, it should be noted that the term “deploy” as used in this patent refers to any adjustment of an implant after the implant has been initially placed into the vetebral space that involves relative motion of one portion of the implant with respect to another portion of the implant. Non-limiting examples of deployment include implants that have one portion that pivots or moves curvilinearly with respect to another portion and implants that have one portion that slides or moves linearly with respect to another portion. Implants that expand in any manner and in any direction fall under the definition of “deploy.” Second it should be noted that the inventor contemplates multiple devices and methods for deploying an implant. While specific embodiments will be described, they should not be understood to be limiting but rather exemplary only.

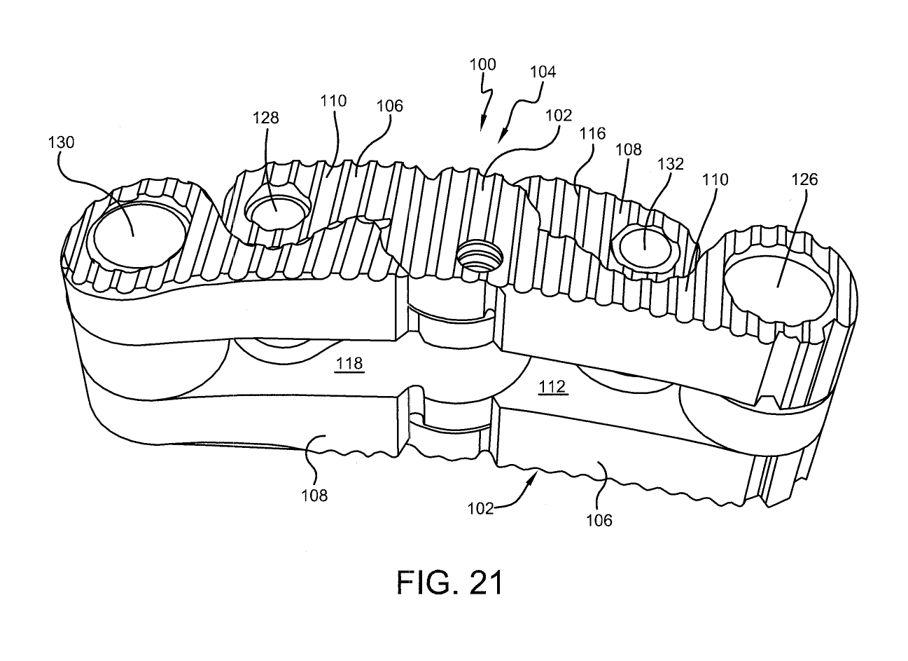

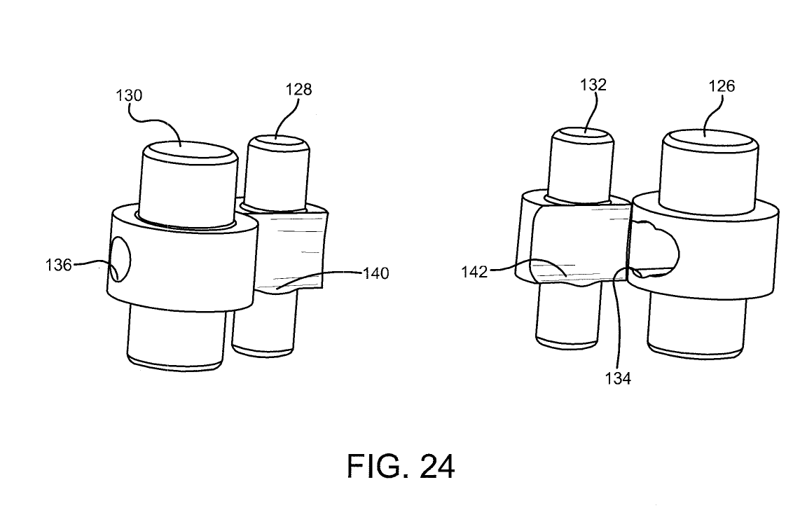

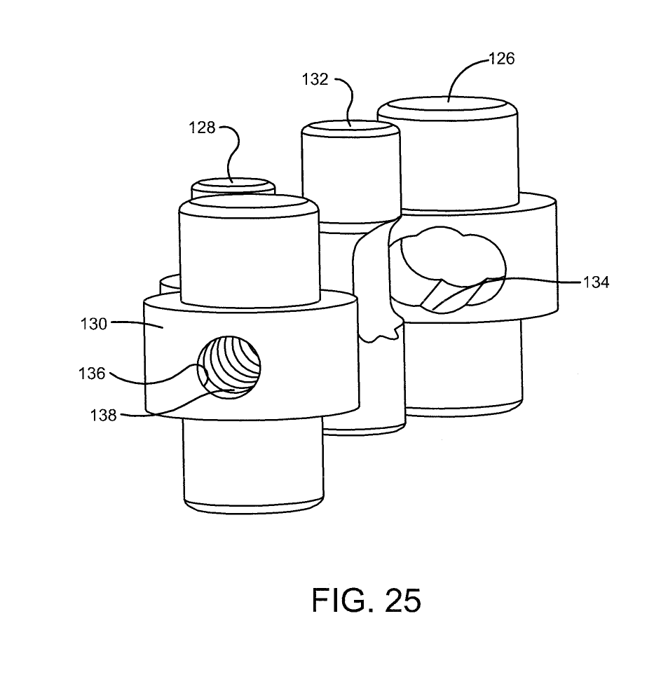

With reference now to FIGS. 1-3 and 21-25, the implant that is deployed according to this invention can be any implant chosen with the sound judgment of a person of skill in the art. For the embodiment shown, the implant 100 may have a pair of contact surfaces 102, 102 that contact the corresponding vertebral endplates within the vertebral space 22. These endplates 102 may be serrated/knurled to facilitate cutting into bony endplates to prevent rotation or expulsion of the device by external rotational or flexion-extension forces. The implant 100 may have a first member 106, a second member 108 and a pivotal connection 104 between the first and second members 106, 108. As a result, the second member 108 can be pivoted with respect to the first member 106 from the non-deployed condition (shown in FIGS. 21-22) to the deployed condition (shown in FIG. 23). For the embodiment shown, the first member 106 has first and second beams 110, 112 and the second member 108 has first and second beams 116, 118. The implant 100 may have first and second posts 126, 128 connecting the first beam 110 of the first member 106 to the second beam 112. Similarly, third and fourth posts 130, 132 may connect the first and second beams 116, 118 of the second member 108. These posts 126, 128, 130, 132 may attach to the beams 110, 112, 116, 118 in any manner chosen with the sound judgment of a person of skill in the art. For the embodiment shown, each post 126, 128, 130, 132 is positioned at the outer ends of each beam 110, 112, 116, 118. This orientation provides maximum compression loading characteristics at the area most likely to carry such a load; namely, at the outer rim of each vertebrae endplate. It should be noted, however, that depending on the particular use, the number of posts used and their positions can be varied in accordance with the load requirements across the beams. To provide a relative location reference for the posts only, it can be noted that the first post 126 is the proximal posterior post, the second post 128 is the distal anterior post, the third post 130 is the distal posterior post, and the fourth post 132 is the proximal anterior post. The posterior posts 126, 130 may have corresponding openings 134, 136 for purposes to be discussed below.

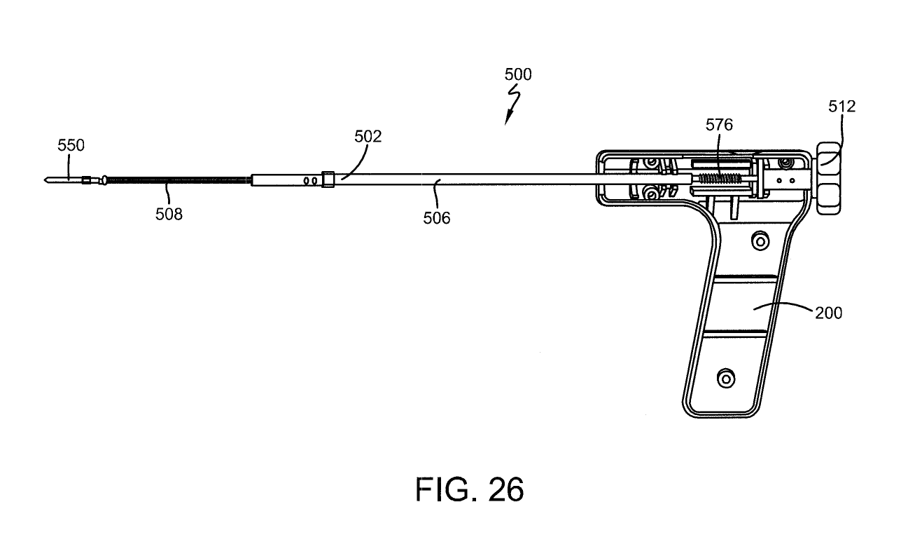

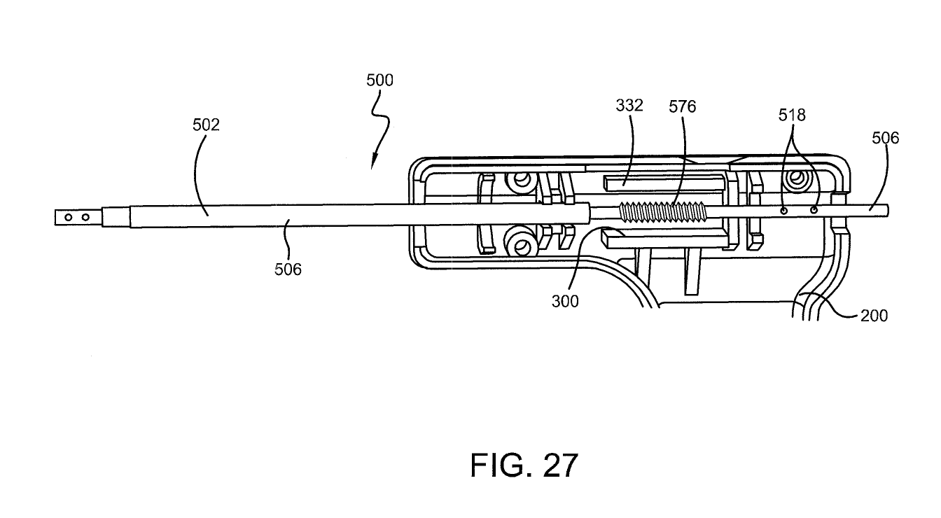

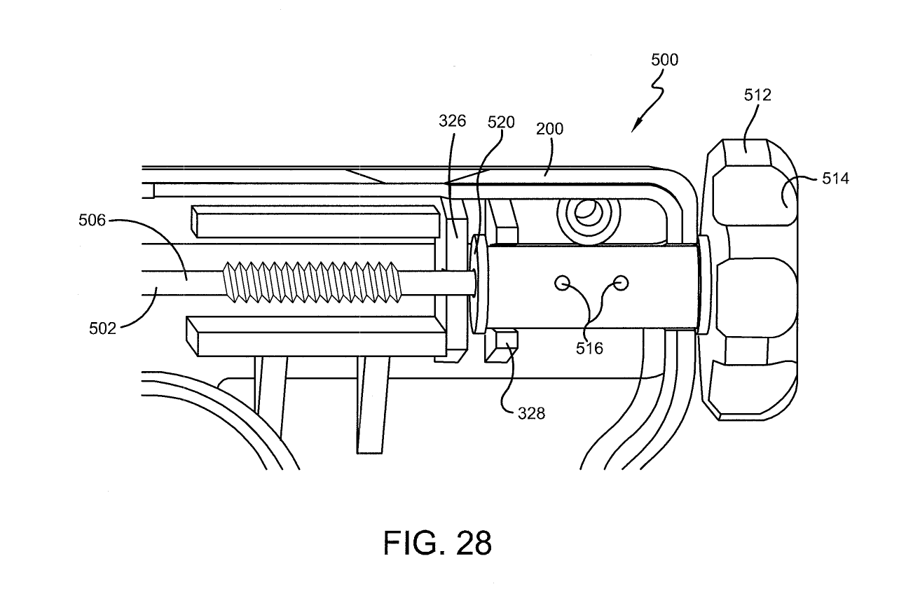

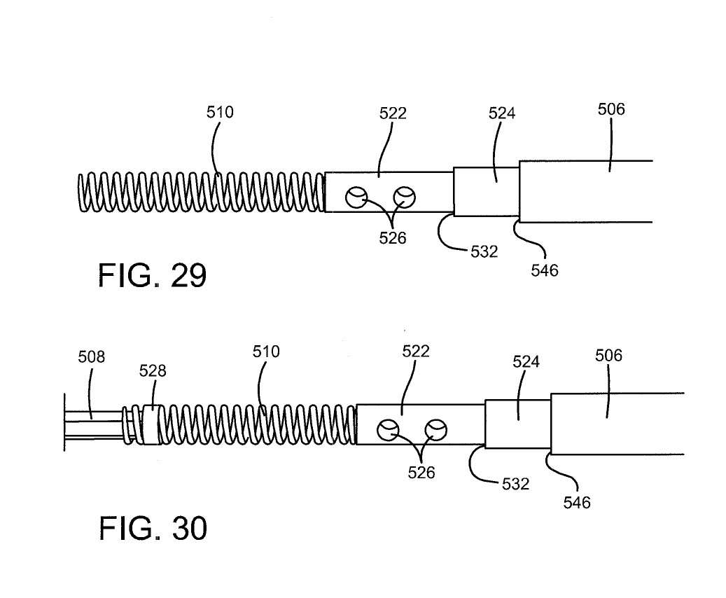

With reference now to FIGS. 3, 5-6, 23 and 26-30, in one embodiment the implant deployment mechanism 500 may include a drive shaft 502 that engages a deployment device 504. When the surgeon rotates the drive shaft 502, it causes the deployment device 504 to deploy the implant 100. While the drive shaft 502 may be constructed in any manner chosen with the sound judgment of a person of skill in the art, for the embodiment shown the drive shaft 502 comprises first and second drive rods 506, 508 interconnected with a compression spring 510. The use of the compression spring 510 between the drive rods 506, 508 provides for increased control for the surgeon. The proximal end of the first drive rod 506 may be received within the handle mechanism 300. For the embodiment shown, the first drive rod 506 is rotatably supported to the handle mechanism 300 within shaft reception tabs 326 formed on the handle members 308, 310. The proximal tip of the first drive rod 506 may extend out of the handle mechanism 300, as shown.

With reference now to FIGS. 3, 5-6 and 26-28, to provide a surface for the surgeon to use when rotating the drive shaft 502, a knob 512 may be attached to the proximal end of the first drive rod 506. The proximal end of the knob 512 may extend outside of the handle mechanism 300 and may have an outer surface 514 that has a textured region to improve the grip for the surgeon. The knob 512 may also have at least one hole 516 (two shown) which are aligned with at least one corresponding hole 518 (two shown) formed in the first drive rod 506. Connectors (not shown) can then be inserted within the holes 516, 518 to attach the knob 512 to the first drive rod 506. The distal end of the knob 512 may have a ridge 520 with an outside diameter greater than the outside diameter of the juxtaposed portion of the knob 512, as shown. This ridge 520 may be positioned on the distal side of a pair of shaft reception tabs 328 formed on the handle members 308, 310, as shown. With this arrangement, the knob 512 (and thus the first drive rod 506) can be rotated with respect to the handle mechanism 300 but cannot move longitudinally with respect to the handle mechanism 300.

With reference now to FIGS. 3, 26-27 and 29-30, the distal end of the first drive rod 506 may contact the proximal end of the compression spring 510, as shown. The distal end of the first drive rod 506 may have first and second reduced diameter portions 522, 524 and may also have at least one hole 526 (two shown). The proximal end of the second drive rod 508 may connect to the distal end of the compression spring 510, as shown. The proximal end of the second drive rod 508 may have a ridge 528 with an outside diameter greater than the outside diameter of the juxtaposed portion of the second drive rod 508 for this purpose. One or more coils of the compression spring 510 may be wrapped around the distal side of the ridge 528, as shown, to attach the spring 510 to the drive rod 508.

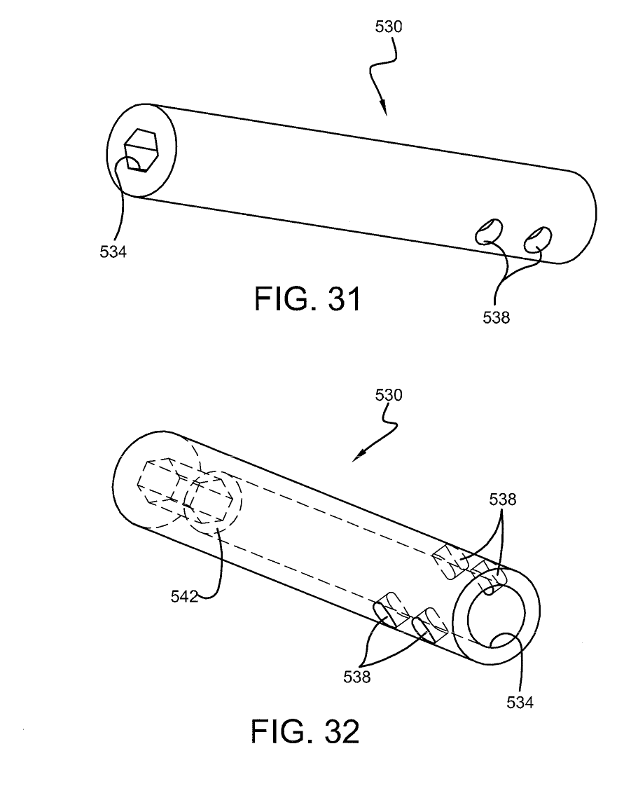

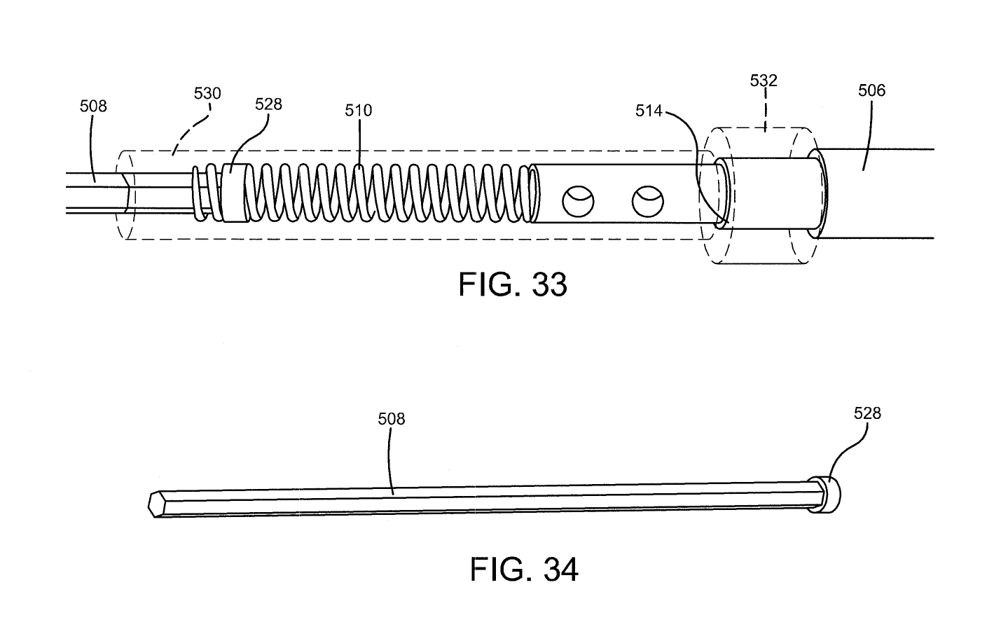

With reference now to FIGS. 3, 26-27 and 29-33, to improve the connection of the first drive rod 506 to the second drive rod 508, a coupling 530 and a bushing 532 may be used. The coupling 530 may be generally cylindrical and may have an opening 534 throughout its length. The opening 534 at the proximal end of the coupling 530 may receive the distal end of the first drive rod 506. The length of the first drive rod 506 received in the opening 534 may be limited by the edge 536 of the first reduced diameter portion 522, as shown. The proximal end of the coupling 530 may have at least one set of holes 538 (two shown) that creates an aperture extending from one outer surface of the coupling 530 to the opposite side of the coupling 530, as shown. The set of holes 538 may be aligned with the hole 526 and a connector (not shown) may be inserted therethrough to connect the coupling 530 to the first drive rod 506. The opening 534 at the distal end of the coupling 530 may receive the proximal end of the second drive rod 508. The spring 510 may be positioned within the opening 534, as shown.

With reference now to FIGS. 3, 26 and 30-34, for reasons discussed below, the second drive rod 508 may have a hexagonal cross-sectional shape. The opening 532 at the distal end of the coupling 530 may have a corresponding hexagonal shape, as shown, to receive the second drive rode 508. The proximal end of the drive rod 508 may have a ridge 528, as noted above, with an outside diameter greater than the outside diameter of the juxtaposed portion of the drive rod 508, as shown. This ridge 528 may be received against a wall 542 formed within the coupling 530 at the point where the opening 534 at the proximal end of the coupling 532 meets the opening 534 at the distal end of the coupling, as shown. In this way, the proximal end of the second drive rod 508 remains attached to the coupling 530. The bushing 532 may have an opening 544 that receives the second reduced diameter portion 524 of the first drive rod 506. The bushing 532 may be positioned between an edge 546 of the second reduced diameter portion 524 and the proximal end of the coupling 530 to prevent unwanted relative motion. As shown in the FIGURES, the drive shaft 502 may be positioned within the openings 410, 414 and 430 in the grip shaft 406, the gripper 408 and the contact member 427, respectively.

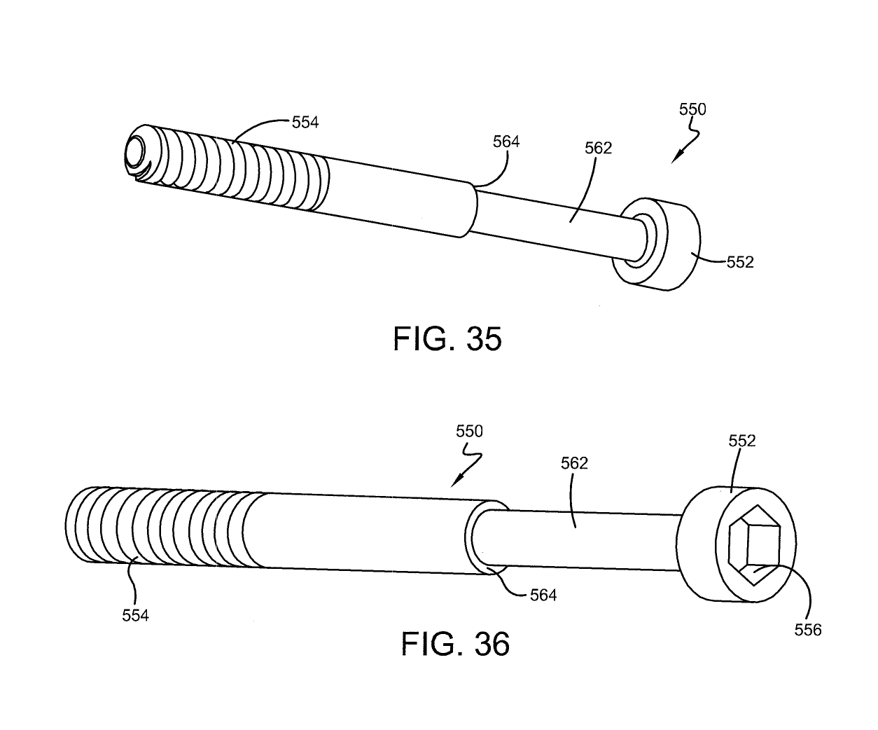

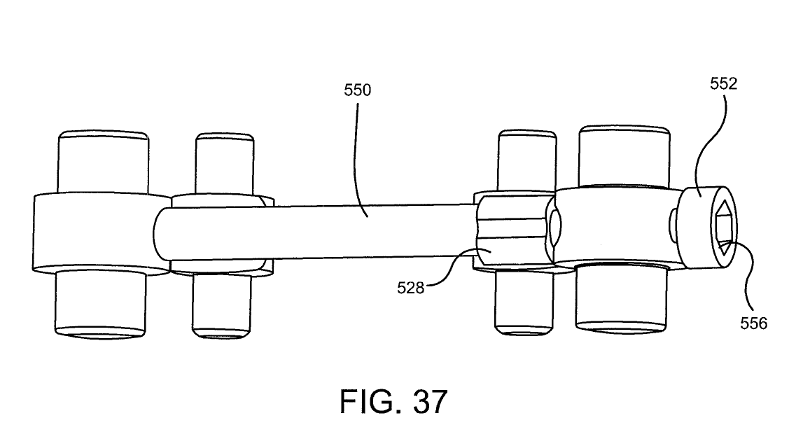

With reference now to FIGS. 3, 22-25, and 34-37, in one embodiment the deployment device 504 includes a screw 550 having a head 552 at its proximal end and a threaded region 554 at its distal end. The screw 550 may be inserted through the opening 134 in the proximal posterior post 126 and then into the opening 136 in the distal posterior post 130. The head 552 may have an engagement surface 556 that engages the distal end of the second drive rod 508. While the engagement of the second drive rod 508 to the screw 550 can be any chosen with the sound judgment of a person of skill in the art, for the embodiment shown, the distal end of the second drive rod 508 may have a hexagonal cross-sectional shape that is received within (engages) a corresponding hexagonal cross-sectional shaped opening that defines the engagement surface 556. The threaded region 554 engages a corresponding threaded region 138 formed on the surface of the distal posterior post 130 that defines, in one embodiment, the opening 136. Thus, rotation of the screw 550 causes the distal posterior post 130 to move along the longitudinal axis of the screw 550.

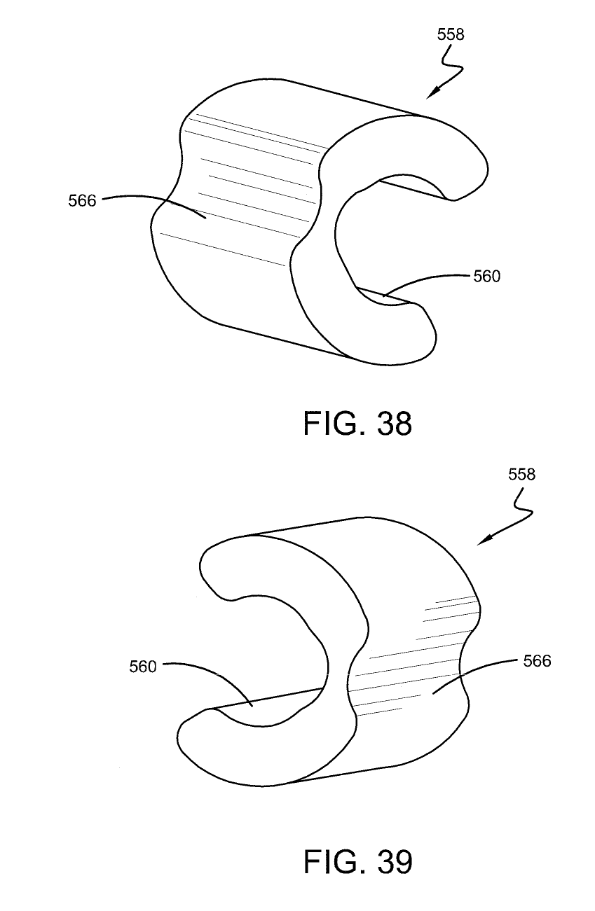

With reference now to FIGS. 3, 22-25, and 34-39, the head 552 of the screw 550 may be larger than the opening 134 in the proximal posterior post 126 to limit the distal movement of the screw 550 with respect to the post proximal posterior post 126 and thus with respect to the implant 100. To help maintain the relative position of the distal posterior post 130 with respect to the screw 550 and the gripper 408, a clip 558 may be used. The clip 558 may be generally C-shaped, as shown in FIGS. 38-39, with an opening 560 that receives a reduced diameter portion 562 of the screw 550. In one specific embodiment, the clip 558 “clips” onto the screw 550 in a known manner. The motion of the clip 558 along the screw 550 may be limited on the distal end of the clip 558 by the edge 564 of the reduced diameter portion 562, as shown. The motion of the clip 558 along the screw 550 may be limited on the proximal end of the clip 558 by the proximal posterior post 126 because the clip 558 is larger than the opening 134. The clip 558 may have a groove 566 defining a pivot point as the clip 558 is pivoted to be received on and pivoted to be removed from the screw 550. Depending on the specific sizes of the screw 550 and the implant 100, the anterior posts 128, 132 may have grooves or cutouts 140, 142, respectively, to receive the screw 550 and/or clip 558.

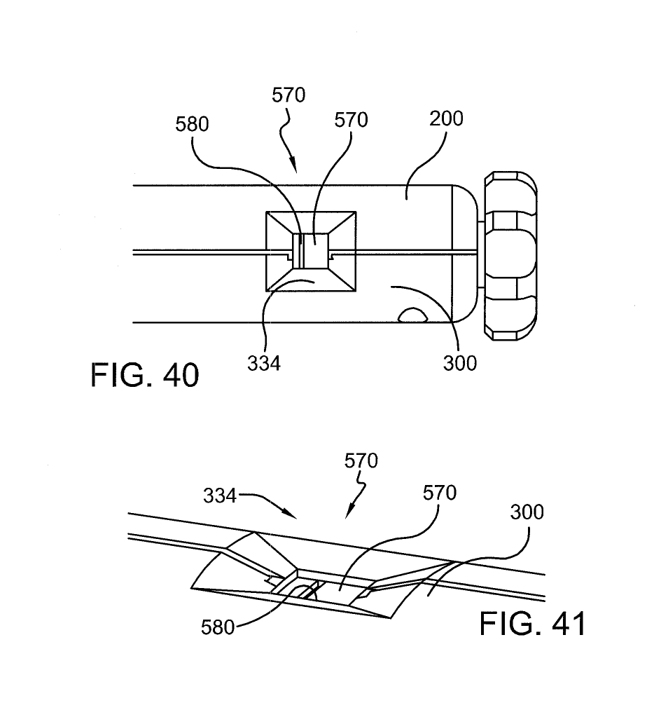

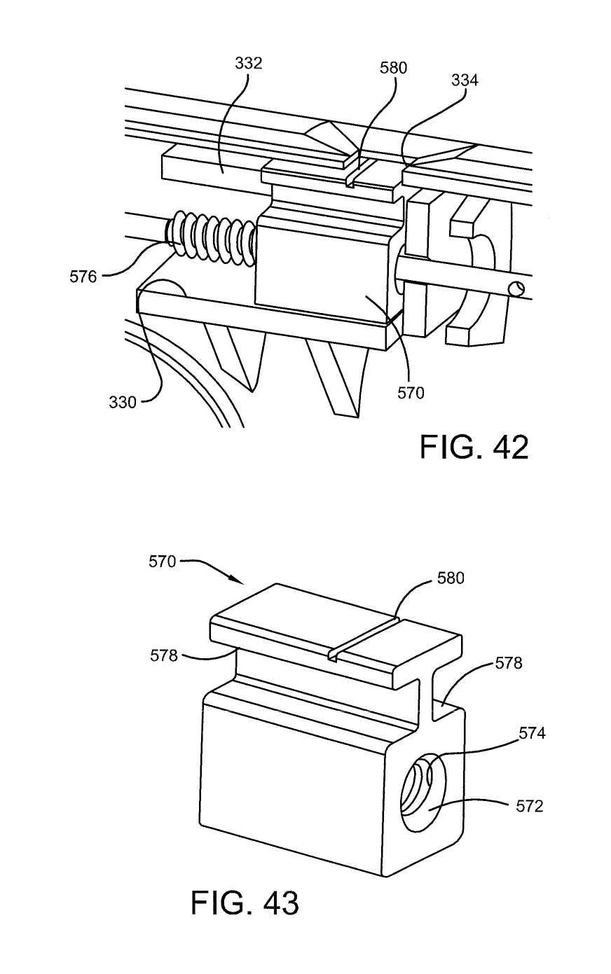

With reference now to FIGS. 3, 5-6, 26-27 and 40-43, in one embodiment a deployment indicator 570 may be used to provide a visual indication to the surgeon regarding the status of implant deployment within the vertebral space. For the embodiment shown, the deployment indicator 570 has an opening 572 defined by a threaded region 574 that receives a corresponding threaded region 576 formed on an outer surface of the drive shaft 502. Thus, as the drive shaft 502 is rotated, the deployment indicator 570 moves along the longitudinal axis of the drive shaft 502. The deployment indicator 570 may be held within the handle mechanism 300. In one embodiment, a lower surface of the deployment indicator 570 rests on, or just above, a support surface 330 that extends from the handle member 308. To maintain the proper orientation of the deployment indicator 570 with respect to the handle mechanism 300, the deployment indicator 570 may have a deployment indicator/inserter rail/groove interconnection that interconnects the deployment indicator 570 and the inserter 200 for relative movement thereby.

With continuing reference to FIGS. 3, 5-6, 26-27 and 40-43, for the embodiment shown, the deployment indicator 570 may have a T-shaped upper portion defining a pair of channels 578, 578 that receive corresponding rails 332, 332 formed on inner surfaces of the handle members 306, 308, as shown. The handle mechanism 300 may have a window 334 through which the deployment indicator 570 can be seen by the surgeon. For the embodiment shown, the portion of the deployment indicator 570 that is visible through the window 334 has an indicator symbol 580. As the indicator symbol 580 moves with respect to the handle mechanism 300, the surgeon can easily determine the degree of implant deployment. In one embodiment, when the indicator symbol 580 is at the proximal end of the window 334, it indicates that the implant 100 has not deployed at all. When the indicator symbol 580 is at the distal end of the window 334, however, it indicates that the implant 100 has fully deployed.

With reference now to FIG. 44 in another embodiment the deployment device 504 includes a cable. The inventor of this patent has disclosed the use of a cable in tension to deploy an implant in, for example, Pub. No. 2007/0073398 tilted SPINE SURGERY METHOD AND IMPLANT and Pub. No. 2009/0270873 tilted SPINE SURGERY METHOD AND INSERTER, both of which are incorporated herein by reference. FIG. 44 shows an inserter 600 for use in inserting a deploying an implant 602. The inserter 600 includes a handle mechanism 604, an implant gripping mechanism 606, and the implant deployment device 504. As this inserter 600 is explained in Pub. No. 2009/0270873, further details will not be provided here. However, it should be noted that the deployment device 504 may include a tension knob 608 that is rotated to apply a tension force to a cable 610 to deploy the implant 602. It should also be noted that the implant 602 is designed similar to the implant 100, described above.

With reference now to FIGS. 44-47, in one embodiment illustrated in FIG. 48, an interference plug 612 may be attached to the distal end of the cable 610 and the proximal end of the cable 610 may be operatively connected to the tension knob 608. The interference plug 612 may be formed of any material, with any size, and with any shape chosen with the sound judgment of a person of skill in the art. It may also be attached to the cable 610 in any manner chosen with the sound judgment of a person of skill in the art. The proximal end of the cable 610 may be first fed through the opening 136 in the distal posterior post 130, then through the opening 134 in the proximal posterior post 126 and then through the inserter 600 up to the tension knob 608. The interference plug 612 is larger than the distal end of the opening 136 in the distal posterior post 130. Thus, when tension is applied at the proximal end of the cable 610, the interference plug 612 contacts the distal posterior post 130 and continued tension applied to the cable 610 causes a tension force to be applied to the distal posterior post 130. The proximal posterior post 126 is simultaneously held by the implant gripping mechanism 606 so the tension force causes the distal posterior post 130 to move toward the proximal posterior post 126 and thus causes the implant 602 to deploy.

With continuing reference to FIGS. 44-47, in one embodiment, the cable 610 may be designed to intentionally fail (break or sever) at a predetermined tension force. The predetermined tension force that causes the cable 610 to fail may be set to occur just after the implant 602 has fully deployed. In one embodiment, the cable 610 has a predetermined failure location 614 for this purpose. The predetermined failure location 614 may be formed by reducing the diameter of the cable 610 or in any other suitable manner. While the predetermined failure location 614 can be positioned at any location on the cable 610 chosen with the sound judgment of a person of skill in the art, in one embodiment the predetermined failure location 614 is located on the proximal side of the interference plug 612 juxtaposed to the interference plug/cable intersection. With this combination of embodiments, after the implant 602 deploys, the cable 610 fails at the predetermined failure location 614. The cable 610 can then be easily removed (it is pulled through the opening 134 in the proximal posterior post 126) and the interference plug 612 remains with the implant 100.

With reference now to FIGS. 44-46 and 48, in another embodiment, the cable 610 similarly has an interference plug 612 and a predetermined failure location 614. But in this case, the cable 610 also has a tail portion 616 that is attached to the distal end of the interference plug 612. The cable 610 is then fed through the implant 602 and inserter 600 as described above. The tail portion 616, however, may also be fed through the inserter 600. After deployment of the implant 602 and after the cable 610 fails at the predetermined failure location 614, the end of the tail portion 616 is pulled by the surgeon to remove the tail portion 616 and the interference plug 612 from the implant 602.

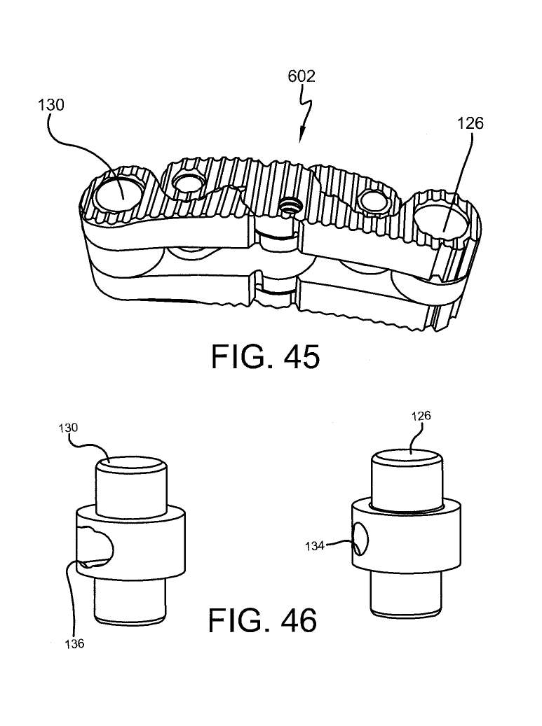

With reference now to FIGS. 45-48, in yet another embodiment, the distal posterior post 130 is rotatable with respect to the beams to which it attaches. As a result, the distal posterior post 130 may rotate as the implant 602 is being deployed to optimize the linear tension on the cable 610 during deployment.

The surgeon may use the inserter 200 or 600 to insert the implant 100 or 602 within the vertebral space 22. To deploy the implant 100, see FIGS. 1-43, the surgeon simply rotates the knob 512 on the inserter 200. This causes the drive shaft 502 to rotate which causes the screw 550 to rotate. As a result, the implant 100 deploys. If a deployment indicator 570 is used, the surgeon can observe the deployment indicator 570 to determine the status of implant 100 deployment within the vertebral space 22. Once the implant 100 has been deployed, the activation member 428 is rotated by the surgeon to cause the contact member 427 to move away from the gripper 408. This causes the gripper 408 to release the implant 100. As the surgeon then begins to pull the inserter 200 away from the implant 100, the distal end of the drive rod 508 disengages from the engagement surface 556 of the screw 550. The inserter 200 can then be easily removed, leaving the deployed implant 100 in its appropriate location in the vertebral space 22.

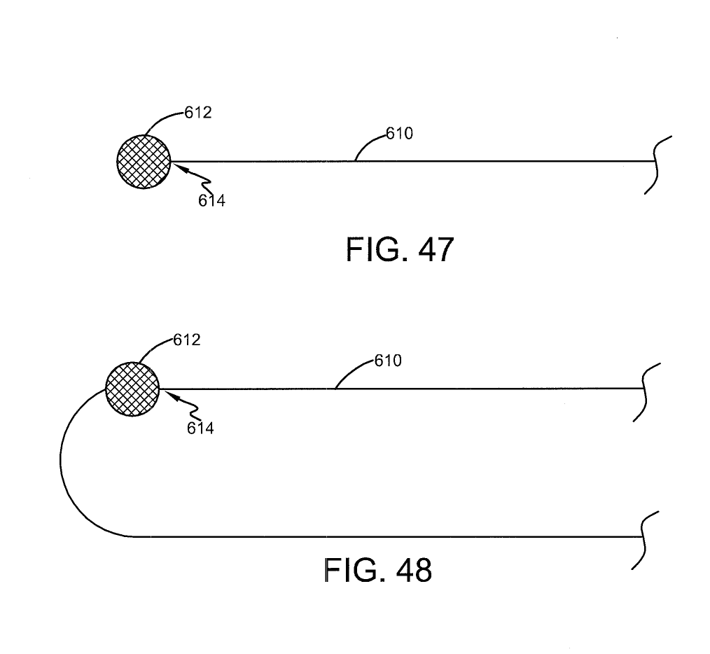

To deploy the implant 602, see FIGS. 44-46, the surgeon simply rotates the knob 608 on the inserter 600. This causes the cable 610 to apply a tension force to the implant 602, to deploy the implant 602. After the implant 602 deploys, the tension force on the cable 610 causes the cable to fail at the failure location 614. If the cable 610 is like the embodiment of FIG. 47, then the surgeon simply operates the implant gripping mechanism 606 to release the implant 602 and then removes the inserter 600 and cable 610 leaving the implant 602 in its appropriate location in the vertebral space 22. If, however, the cable 610 is like the embodiment of FIG. 48, after the cable fails at the failure location 614, the surgeon pulls the tail portion 616 and the interference plug 612 away from the implant 602. The surgeon then simply operates the implant gripping mechanism 606 to release the implant 602 and then removes the inserter 600, the cable 610 and the tail 616 with the interference plug 612 leaving the deployed implant 602 in its appropriate location in the vertebral space 22.

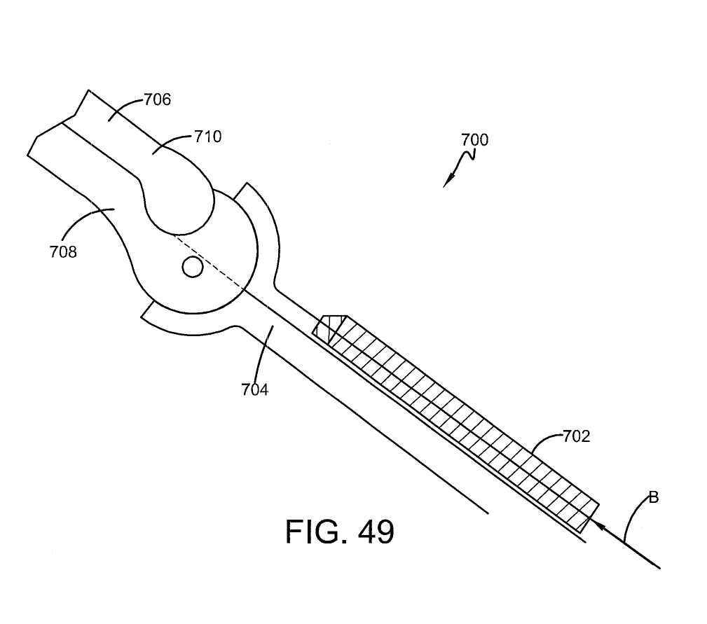

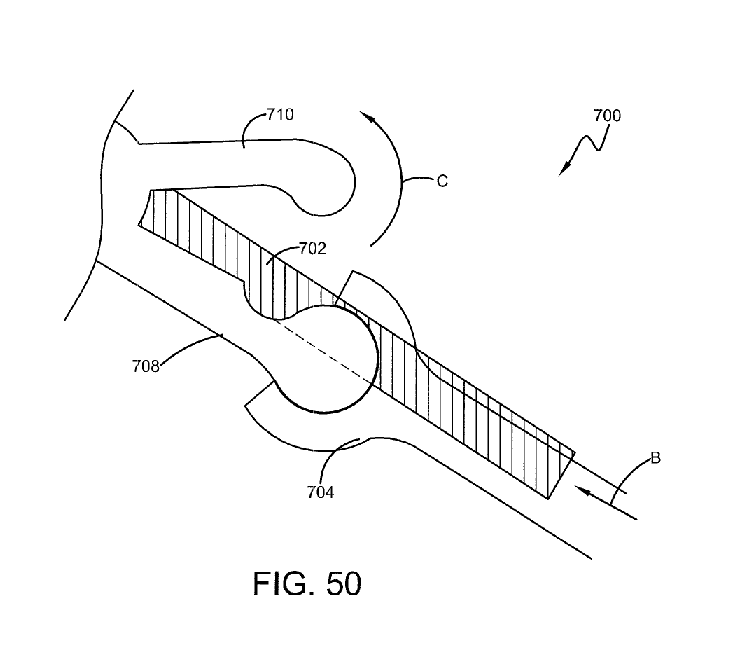

With reference now to FIG. 49-50, in another embodiment a gripper 704 grips a portion of an implant 706 similar to the embodiments discussed above. A deployment mechanism 700, however, does not include a screw or a cable but rather a push rod 702 that may be extended from the inserter into physical contact with the implant 706 to deploy the implant 706. While the implant used with a push rod can be any chosen with the sound judgment of a person of skill in the art, the implant 706 shown is similar to implants described above and includes a second member 710 that can be pivoted in direction C with respect to a first member 708 from a non-deployed condition (shown in FIG. 49) to a deployed condition (shown in FIGS. 50 and 53). The push rod 702 can be of any size and shape chosen with the sound judgment of a person of skill in the art. In one embodiment, the push rod 702 is a solid member. In another embodiment, the push rod 702 is formed out of a series of linked segments. In yet another embodiment, the push rod 702 is flexible and has shape memory such that when it exits the confines of the inserter it moves along a predetermined radius of curvature against the implant 706.

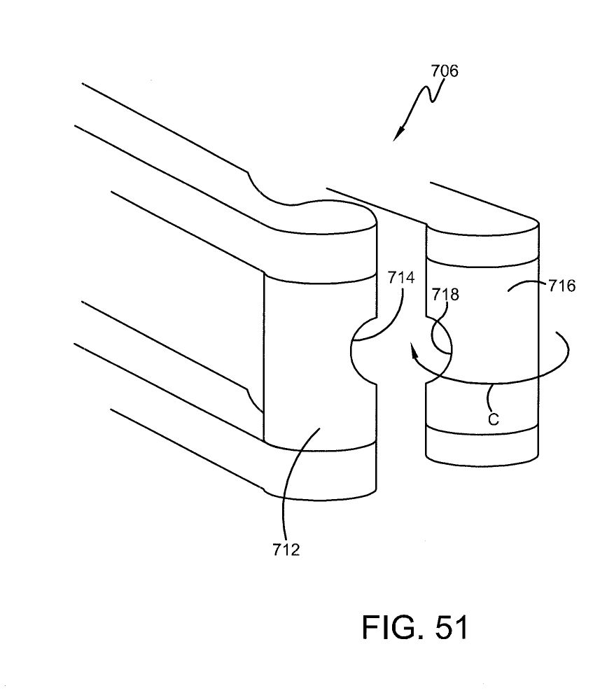

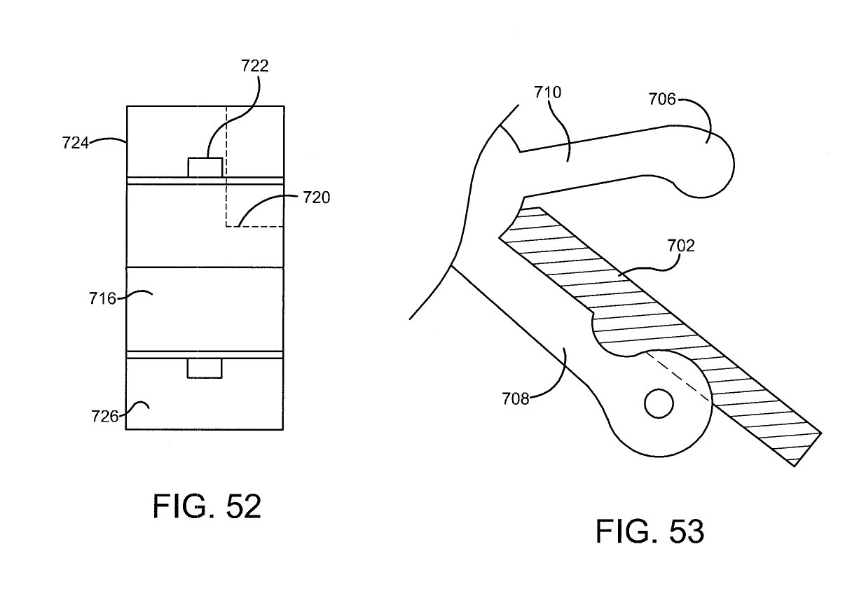

In one embodiment, shown in FIG. 51, implant post 712 has a groove or cutout 714 on its anterior surface and implant post 716 has a groove or cutout 718 on its posterior surface. The push rod 702 can them be extended along the gripper 704 and then within the cutout 714. Continued extension of the push rod 702 in the distal direction B causes the push rod 702 to contact the post 716 via the cutout 718. Further extension of the push rod 702 deploys the implant 706. In another embodiment, shown in FIGS. 52-53, there is a groove or cutout 720 on the anterior surface of implant post 716 juxtaposed to a groove or cutout 722 on either the superior limb 724 and/or the inferior limb 726. For this embodiment the push rod 702 is extended along the gripper 704 and then within the cutouts 720, 722. Continued extension of the push rod 702 in the distal direction causes the push rod 702 to contact the post 716 and further extension of the push rod 702 deploys the implant 706.

With reference now to FIG. 49-50, it should be noted that the push rod 702 can be positioned in any manner chosen with the sound judgment of a person of skill in the art. In one embodiment, the push rod 72 extends from inside the inserter similar to the embodiments discussed above. In another embodiment, the push rod 702 extends along a track formed on the outside surface of the inserter. In yet another embodiment, the push rod 702 is a separate instrument that does not engage the inserter for relative motion.

Numerous embodiments have been described, hereinabove. It will be apparent to those skilled in the art that the above methods and apparatuses may incorporate changes and modifications without departing from the general scope of this invention. It is intended to include all such modifications and alterations in so far as they come within the scope of the appended claims or the equivalents thereof.

Having thus described the invention, it is now claimed:

1. A device comprising:
an inserter for use in inserting an associated implant into a vertebral space, the inserter comprising: a handle for use by a surgeon;
an implant gripping mechanism supported to the inserter and adjustable by the surgeon to grip and to release the associated implant within the vertebral space;
an implant deployment mechanism supported to the inserter and adjustable by the surgeon to deploy the associated implant within the vertebral space; and,
a deployment indicator supported to the inserter that provides a visual indication to the surgeon regarding the status of implant deployment within the vertebral space.

2. The device of claim 1 wherein:
the inserter comprises: a deployment indicator/inserter rail/groove interconnection that interconnects the deployment indicator and the inserter for relative movement thereby; and,
the relative movement indicates the status of implant deployment.

3. The device of claim 2 wherein the inserter has a window through which the deployment indicator can be seen by the surgeon.

4. The device of claim 1 wherein the deployment indicator indicates the degree of implant deployment.

5. The device of claim 1 wherein the deployment indicator indicates both no implant deployment and full implant deployment.

6. The device of claim 1 wherein:
the implant deployment mechanism comprises:
a drive shaft that can engage the associated implant; and,
a deployment device that can rotate the drive shaft to cause the associated implant to deploy; and,
the deployment indicator is operatively attached to the drive shaft.

7. The device of claim 6 wherein:
the drive shaft comprises a threaded region;
the deployment indicator has a threaded region that engages the threaded region of the drive shaft; and,
the deployment indicator moves relative to the inserter as the drive shaft is rotated.

8. A method comprising the steps of:
(A) providing an implant made to be placed into a vertebral space and comprising a first member and a second member movably attached to the first member;
(B) providing a surgical inserter;
(C) providing a cable operatively attached to the surgical inserter and operatively attached to at least one of the first or second members;
(D) preparing the vertebral space to receive the implant;
(E) inserting the implant into the vertebral space in a non-deployed condition; and,
(F) applying tension to the cable to:
(1) move the second member with respect to the first member to deploy the implant within the vertebral space; and,
(2) intentionally cause the cable to fail.

9. The method of claim 8 wherein:
step (C) comprises the step of: providing the cable with a predetermined failure location; and,
step (F)(1) comprises the step of: causing the cable to fail at the failure location.

10. The method of claim 8 further comprising the step of:
removing the cable from the implant.

11. The method of claim 8 wherein:
step (A) comprises the step of: providing the implant with a first opening;
step (B) comprises the step of: providing the surgical inserter with an implant deployment mechanism;
step (C) comprises the step of: providing the cable with a first portion that is extended within the first opening and an interference plug that cannot be extended through the first opening;
step (F) comprises the step of: using the implant deployment mechanism to apply tension to the cable;
step (F)(1) comprises the steps of:
(a) sliding the cable within the first opening until the interference plug contacts the implant juxtaposed to the first opening; and,
(b) moving the second member with respect to the first member only while the interference plug is in contact with the implant juxtaposed to the first opening.

12. The method of claim 11 wherein:
step (C) comprises the steps of:
providing one end of the cable with a tail; and,
operatively attaching both the cable and the tail to the surgical inserter;
step (F) comprises the steps of:
applying tension to the cable;
causing the cable to fail thereby separating the tail from the cable; and,
the method further comprises the steps of:
removing the cable from the implant; and,
removing the tail from the implant.

13. The method of claim 8 wherein:
step (A) comprises the step of: providing the implant with a post that is rotatable with respect to the implant; and,
step (F)(1) comprises the step of: causing the post to rotate with respect to the implant to optimize the linear tension on the cable during deployment.

14. A surgical system comprising:
an implant made to be placed into a vertebral space and comprising: a first member and a second member movably attached to the first member;
an inserter for use in inserting the implant into the vertebral space, the inserter comprising: a handle mechanism for use by a surgeon;
an implant gripping mechanism supported to the handle mechanism and adjustable by the surgeon to grip and to release the implant within the vertebral space; and,
an implant deployment mechanism supported to the handle mechanism and comprising: a drive shaft that: is attachable to the implant; and, can be rotated by the surgeon to deploy the associated implant within the vertebral space by moving the first member of the implant with respect to the second member of the implant.

15. The surgical system of claim 14 wherein:
the implant gripping mechanism comprises:
(a) a gripper having: an opening; and, a pair of arms;
(b) a grip activator comprising: a contact member; and, an activation member;
(c) wherein the activation member can be rotated in a first direction to cause the contact member to contact the gripper to cause the arms to move toward each other to grip the implant; and,
(d) wherein the activation member can be rotated in a second direction to cause the contact member to move out of contact with the gripper to cause the arms to move away from each other to release the implant; and,
the drive mechanism comprises a drive shaft that: is received within the opening in the gripper; and, can be rotated by the surgeon to deploy the implant within the vertebral space.

16. The surgical system of claim 14 wherein:
the first member of the implant comprises a first opening having a threaded region;
the second member of the implant comprises a second opening;
the drive mechanism comprises:
(a) a drive shaft that: has a first end operatively attached to the handle mechanism; and, has a second end; and,
(b) a screw received within the second opening and having a first end with an engagement surface that engages the second end of the drive shaft and a second end with a threaded region that engages the threaded region of the first opening; and,
wherein rotation of the drive shaft causes: the screw to rotate; and, the implant to deploy within the vertebral space.

17. The surgical system of claim 16 wherein the drive shaft comprises:
a first drive rod having a first end that is supported to the handle mechanism and a second end;
a spring having a first end operatively attached to the second end of the first drive rod and a second end; and,
a second drive rod having a first end operatively attached to the second end of the spring and a second end that defines the second end of the drive shaft.

18. The surgical system of claim 17 wherein the drive mechanism comprises:
a knob that is: fixedly attached to the first end of the first drive rod; and, rotatably attached to the handle mechanism; and,
wherein rotation of the knob with respect to the handle mechanism causes: the drive shaft to rotate with respect to the handle.

19. The surgical system of claim 17 further comprising:
a coupling having: a first end that attaches to the second end of the first drive rod; a second end that attaches to the first end of the second drive rod; and, an opening that receives the spring.

20. The surgical system of claim 16 wherein:
the second end of the drive shaft has a hexagonal cross-section and the engagement surface of the screw has a corresponding hexagonal cross-section.
